(19) United States
(12) Patent Application Publication
(10) Pub. No.: US 2007/0184501 A1
(43) Pub. Date: Aug. 9, 2007

(54) Blocker reagent for reduction of heterophile interferences

(75) Inventor: Bruce H. Odegaard, Crystal, MN (US)

Correspondence Address: BECKMAN COULTER, INC., P.O. BOX 169015, MAIL CODE 32-A02, MIAMI, FL, 33116-9015, US

(73) Assignee: Beckman Coulter, Inc., Fullerton, CA (US)

(21) Appl. No.: 11/349,444

(22) Filed: Feb. 7, 2006

(51) Int. Cl. G01N 33/53

(52) U.S. Cl. 435/7.92

(57) ABSTRACT

A specific blocking reagent, method of manufacture thereof and methods of use are provided. The blocking reagent uses the specific antibody structure recognized by the target, e.g., analyte, and any interfering substances, e.g., heterophile molecules. The blocking reagent is, however, an inactivated form of the target-specific antibody. Thus, the blocking reagent cannot bind the target because the target-binding regions have been inactivated. The blocking reagent can, however, block interfering substances such as heterophile molecules as these binding regions remain active. In this manner, the blocking reagent provides highly specific removal of interferents. The blocking reagent thus has broad applicability in immunoassays as well as enhanced delivery of therapeutic and/or diagnostic agents.

### BACKGROUND OF THE INVENTION

1. Field of the Invention

The present invention relates generally to reagent, method of manufacture and method of use for specifically reducing interference by a substance with a target. More specifically, reagents and methods are provided that utilize substantially identical epitopic structures recognized by a particular interferent, e.g., heterophiles, that are incapable of binding the analyte of interest to obtain analyte-specific reduction of heterophile interference in immunoassays. The reagent and methods also are applicable in improving the specific delivery of diagnostic and/or therapeutic agents.

2. Description of the Related Art

Immunoassays are assay reagent systems that exploit the ability of an antibody to specifically recognize and bind to a particular analyte or “antigen.” An antigen is a substance which is capable of inducing an immune response, i.e., antibody production, when introduced into an animal or human body. The particular region of an antigen or analyte that is recognized by an antibody, and to which the antibody binds, is referred to as an “epitope” or binding region.

The simplest immunoassay reagent system involves merely incubating an antibody that is capable of binding to a target molecule (i.e., the “analyte”) with a sample that is suspected to contain the analyte. The presence of the target molecule is determined by the presence, and is proportional to the concentration, of any immune complexes that form through the binding of the antibody and the analyte. In order to facilitate the separation of such immune complexes from uncomplexed material, a solid phase is typically employed. In more sophisticated immunoassays such as particle-enhanced assays, the concentration of the target molecule is determined by binding the antibody to a support such as latex particles, and then incubating the support-bound antibody in the presence of the analyte-containing sample.

Target molecules that have become bound to the immobilized antibody can be detected in a variety of ways. For example, the support can be incubated in the presence of a labeled, second antibody (i.e., a “sandwich” immunoassay) that is capable of binding to a second epitope of the target molecule. Binding of the labeled antibody on the support thus requires the presence of the target, and is proportional to the concentration of the target in the sample.

Alternatively, the sample is incubated with a known amount of labeled target molecule and antibody binding sites. Any target molecules in the sample compete with the labeled target molecules for the antibody binding sites. Thus, the amount of labeled target molecules that are able to bind the antibody is inversely proportional to the concentration of target molecules in the sample. This is known as a competitive immunoassay.

The various immunoassay formats can be further divided into two main classes, depending upon whether the assay requires the separation of bound species from unbound species. Heterogeneous immunoassays require such separation, and hence entail a separation or isolation step. In contrast, homogeneous assays are designed such that the removal of bound species from unbound species is unnecessary. Because homogeneous assays lack a separation step, and are more easily automated, they are more desirable than heterogeneous assays in applications that require the screening of large numbers of patients.

In particle-enhanced immunoassays, an immune complex formation caused by a reaction between one or more particle-bound antibodies and the analyte results in particle aggregation. If the immune complex is large enough, it will become capable of scattering light, or of spontaneously precipitating. In such cases, agglutination, nephelometric, or turbidimetric detection methods may be employed. Nephelometric methods measure the light scattered by a suspension of particles or reflected toward a detector that is not in the direct path of light. In contrast, turbidimetric methods measure the reduction of light transmitted through the suspension of particles or aggregates. The reduction is caused by reflection, scatter, and absorption of the light by the aggregates. Agglutination assays measure the precipitation of antibody-antigen complexes. Such assays can be extremely sensitive, and are amenable to automation. Because nephelometric and turbidimetric methods do not require the separation of the initially present antibody from the immune complexes formed in the assay, such assays are homogenous immunoassays.

Heterophile molecules are a well-known cause of interference in all immunoassay types and formats and are present in up to 40% of normal blood donors. Heterophile molecules are produced against poorly defined antigens and generally show weak avidity and may be multispecies specific. Heterophiles may be IgAs, IgEs, IgGs, IgMs or Ig(n) and may be poly- or mono-specific.

Heterophile antibodies may interfere in two-site immunoassays by causing false-positive results. This form of interference results from the heterophile molecule bridging the capture and signal antibodies, in the presence or absence of the target analyte, yielding a false-positive result. Alternatively, heterophile molecules may cause false-negative results by binding directly with the capture antibody, thereby blocking the capture antibody's reactive site from binding the target analyte. Either type of interference results in decreased sensitivity or specificity of the affected immunoassay.

Current approaches to reducing heterophilic interference in immunoassays focus on flooding the patient sample and/or subsequent immunochemical reaction mixture with a broad range of non-specific “normal IgG” antibodies, or in some cases monoclonal IgG antibody of the same isotype. These approaches simply rely on the hope that within the broad range of IgG antibody applied to the immunoassay enough specific epitopes will be present to bind enough heterophile molecules in the patient sample to allow for a sufficient sensitivity or specificity for the particular assay. Thus, current heterophile antibody “blocking” approaches do not specifically target the heterophile antibody that may interfere with a particular immunoassay reagent system. Such an approach is needed to allow more sensitivity or specificity in immunoassay test results.

The problem described above is not confined to immunoassay interference by heterophile molecules. For example, monoclonal antibodies have been used to enhance delivery of diagnostic and/or therapeutic agents to assess, diagnose, treat and/or prevent disease or a particular condition. By way of example, immunoconjugates, comprising an anticancer agent conjugated to a monoclonal antibody that is specific for the particular type of cancer cells at issue, may be prepared and administered to the patient by known routes and methods. Significantly, immunoconjugates may comprise a number of diagnostic and/or therapeutic agents as readily recognized by those skilled in the art. Typical agents used against cancer cells are radioactive isotopes, drugs, toxins and enzymes.

Attempts to improve the efficacy of the exemplary anticancer immunoconjugate have been made by administering a protective dose of non-specific blocking antibodies capable of non-specific and/or cross-reactive binding with non-target cells, tissues and/or antigens encountered along the therapeutic pathway, together with the immunoconjugate comprising antibodies specific with the target cells or tissues. See, e.g., U.S. Pat. No. 5,648,059 to Gray, incorporated herein in its entirety by reference. In this context, the non-target interfering cells, tissues and/or antigens may comprise cells, combinations of cells, tissues, amino acids or functional constituent or derivative thereof, polypeptides, interfering antibodies, e.g., heterophile antibodies, and the like. This prior art protective dose is designed to ensure to the extent possible, maximum dosage delivery of the active immunoconjugate agent at the target cell or tissue, by minimizing inactivating binding, interference, and or non-target retention of the immunoconjugate. The protective effect is, however, minimized due to the non-specific nature of the approach.

Known blocking antibodies used in the prior art protective dose may be administered prior to the active therapeutic and/or diagnostic agent or concurrently. If used as a component of a delivery vehicle for diagnostic or therapeutic immunoconjugates or other agents, known non-specific blocking antibodies may be associated with the active agent by any method known to those skilled in the art. By way of example, the active agent may be combined with the non-specific blocking antibody. One skilled in the art will readily recognize additional technologies for combining a diagnostically and/or therapeutically active agent with such blocking antibodies.

The non-specific nature of the blocking antibodies used in prior art protective dosing results in an inability to predict efficacy of the protective dose and, as a result, an inability to accurately predict the active agent's dose that is actually delivered to the target. Thus, it is desirable to design and deliver blocking antibodies that specifically mimic the carrier as protection against unknown non-target cells, tissues, antigens and/or antibodies or antibody fragments to enhance therapeutic dosing of immunoconjugate agents or other therapeutic agents susceptible to the effects of non-target binding.

### BRIEF SUMMARY OF THE INVENTION

A specific blocking reagent, method of manufacture thereof, methods of use, and an immunoassay reagent system and method are provided. The blocking reagent uses the specific antibody structure that both recognizes the target, e.g., analyte, and is recognized by any interfering substances, e.g., heterophile molecules. The blocking reagent is, however, a partially inactivated form of the target-specific antibody. Thus, the blocking reagent cannot bind the target because the target-binding regions have been specifically inactivated. The blocking reagent can, however, bind interfering substances such as heterophile molecules as these binding regions remain active. In this manner, the blocking reagent provides highly specific protection from interferents. The blocking reagent thus has broad applicability in immunoassays as well as enhanced delivery of therapeutic and/or diagnostic agents.

An object of the present invention is to provide a method of manufacturing a specific blocker reagent.

Another object of the present invention is to provide a blocker reagent that will specifically bind with and block interfering substances such as heterophile molecules contained in a patient sample.

Another object of the present invention is to provide an immunoassay reagent system comprising a specific blocker reagent designed to specifically bind and block heterophile molecules from interfering with the immunoassay.

Another object of the present invention is to provide an immunoassay method that utilizes a specific blocker reagent to bind with and block heterophile molecules that may be contained in the patient test sample from interfering with the immunoassay.

Another object of the present invention is to provide a method for improving delivery of agent(s) to a target in a subject by administering a specific blocker reagent to bind with and block specific interfering substances, e.g., non-target cells, tissues and/or antigens encountered along the therapeutic pathway.

Another object of the present invention is to provide a pharmaceutical composition to improve delivery of diagnostic and/or therapeutic agents to a target in a subject.

The figures and the detailed description which follow more particularly exemplify these and other embodiments of the invention.

### BRIEF DESCRIPTION OF THE DRAWINGS

The invention may be more completely understood in consideration of the following detailed description of various embodiments of the invention in connection with the accompanying drawings, which are as follows.

FIG. 1 is an illustration of a typical prior art sandwich immunoassay.

FIGS. 2A and 2B illustrate the effects of heterophile interference in prior art assays.

FIG. 3 is an illustration of successful prior art heterophile blocking technique.

FIG. 4 illustrates failure of prior art heterophile blocking technique.

FIG. 5 is a flowchart for a method of manufacturing one embodiment of the invention.

FIG. 6 illustrates one embodiment of the exemplary manufacturing method and resulting product.

FIG. 7 provides illustration of one embodiment of the invention used in an immunoassay.

### DETAILED DESCRIPTION OF THE INVENTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

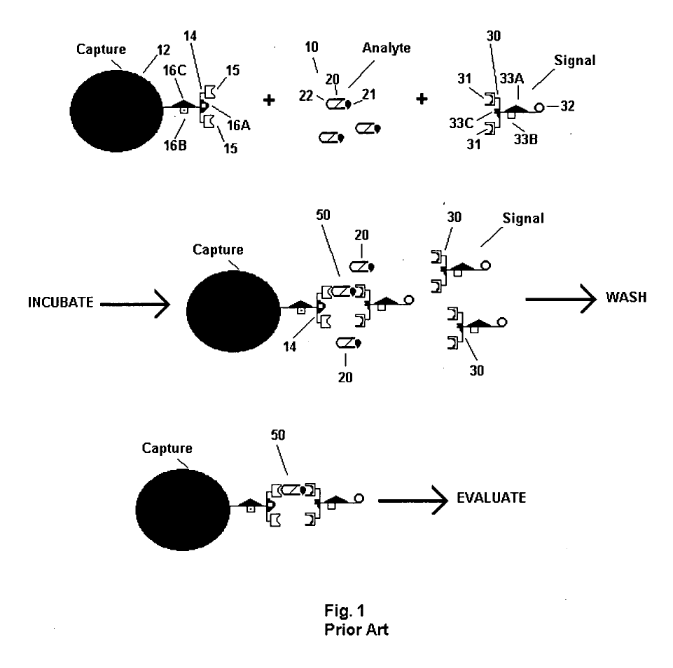

Referring now to FIG. 1, a typical sandwich-type immunoassay 10 is provided. Thus, a paramagnetic particle 12 is illustrated supporting a target-specific capture antibody 14 and mixed with a patient sample containing the target, e.g., an analyte 20 and a target-specific signal antibody 30.

The capture antibody comprises at least one target, e.g., analyte, binding region 15, and at least one heterophile interferent binding region 16A, 16B, 16C. Each of the binding regions are active in FIG. 1, meaning that each analyte binding region 15 is capable of binding analyte 20 and each heterophile interferent binding region 16A, 16B, 16C is capable of binding heterophile interferents. The target-specific binding regions 15 are shown having the same structural shape in the Figure, indicating for descriptive and illustrative purposes that the two binding regions 15 shown have an affinity and are capable of binding a specific epitope on one particular target analyte. Similarly, the target-specific signal antibody is illustrated with two target binding regions 31 having the same structural shape, indicating affinity and a capability for binding an epitope on one specific target analyte.

Moreover, the heterophile binding regions 16A, 16B, 16C on the target-specific capture antibody 14, are shown having differing structural shapes, indicating an affinity and a capability for being bound by heterophile molecules having structural features corresponding with, and complementary to, the heterophile binding regions 16A, 16B, 16C. Similarly, the target-specific signal antibody 30, is shown with heterophile binding regions, 33A, 33B and 33C, each having an affinity and a capability for being bound by heterophile molecules having structural features correspondent with binding regions 33A, 33B or 33C, respectively.

The signal antibody 30 further comprises a signal generating label 32 that is conjugated to the signal antibody 30 by known methods.

Accordingly, the target or analyte 20 is shown with two binding regions, or epitopes. The first binding region 22 has an affinity for binding within the at least one analyte binding region 15 on the capture antibody 14. The second binding region or epitope 21 has an affinity. for binding with the at least one corresponding analyte binding region 31 on the signal antibody 30.

The paramagnetic particle-supported target-specific antibody 14, analyte 20, and target-specific signal antibody 30 are incubated under time and temperature conditions sufficient to allow binding or complexing to occur. In this exemplary embodiment, there are no interfering substances, e.g., heterophile molecules. Thus, complex 50 forms as desired, comprising the paramagnetic particle-supported specific antibody 14 bound with the analyte 20 which is in turn bound with the signal antibody 30. Structurally, the analyte binding region 15 of the capture antibody 14 is bound with epitope 22 of the analyte while epitope 21 of the analyte is bound with binding region 31 of the signal antibody, forming complex 50.

Following incubation, the mixture is washed and all unbound material is eliminated, i.e., any free analyte 20 and/or free signal antibody 30. The material bound to the paramagnetic particle-supported specific antibody 14, i.e., complex 50, is not washed away and is evaluated by any number of known means, e.g., chemiluminescence, to generate a measurable signal and provide either a qualitative or quantitative result for the analyte and with an inherent specificity and sensitivity, terms well known to those skilled in the art.

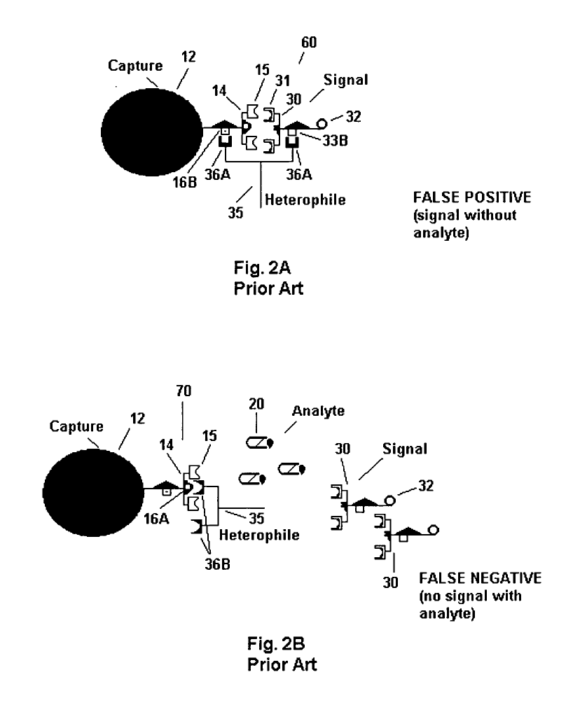

Turning to FIGS. 2A and 2B, prior art immunoassays, such as those discussed above in connection with FIG. 1, are subject to interference by a variety of interfering substances, e.g., heterophilic molecules. Heterophiles are capable of interfering with assays in two basic ways: false positive results as illustrated in FIG. 2A and false negative results as shown in FIG. 2B.

FIG. 2A thus illustrates a false positive interferent complex 60 comprising the paramagnetic particle-supported specific antibody 14 and the signal antibody 30 bridged and bound by a heterophile molecule 35. In this exemplary embodiment of the false positive complex 60, the heterophile binding region 16B of the capture antibody 14 and the heterophile binding region 33B of the signal antibody 30 have sufficiently similar structural features such that both are bound by the heterophile binding regions 36A. It is particularly noteworthy that there is no analyte in this particular illustration; thus a negative signal is expected.

However, as seen in FIG. 2A, this form of heterophile interference results in an effective bridging of the specific antibody 14 and the signal antibody 30 including signal 32, in the absence of any analyte. Accordingly, evaluation of this false positive complex 60 generates a positive signal, falsely indicative of the presence of analyte.

Alternatively, as illustrated in FIG. 2B a false negative interferent complex 70 may form, comprising the paramagnetic particle-supported specific antibody 14 and a heterophile antibody 35 bound thereto. In this case, one of the heterophile binding regions 36B is bound to the capture antibody heterophile binding region 16A, preventing the analyte 20 present in the patient sample from binding to the analyte binding region 15 and providing a falsely negative signal.

This falsely negative interferent complex 70 is formed not only to the exclusion of the analyte 20 but also the signal antibody 30. Thus, upon washing, the interferent complex 70 remains and the unbound analyte 20 and unbound signal antibody 30 with label 32 are both eliminated. Evaluation of complex 70 provides a falsely negative signal, and a false negative response.

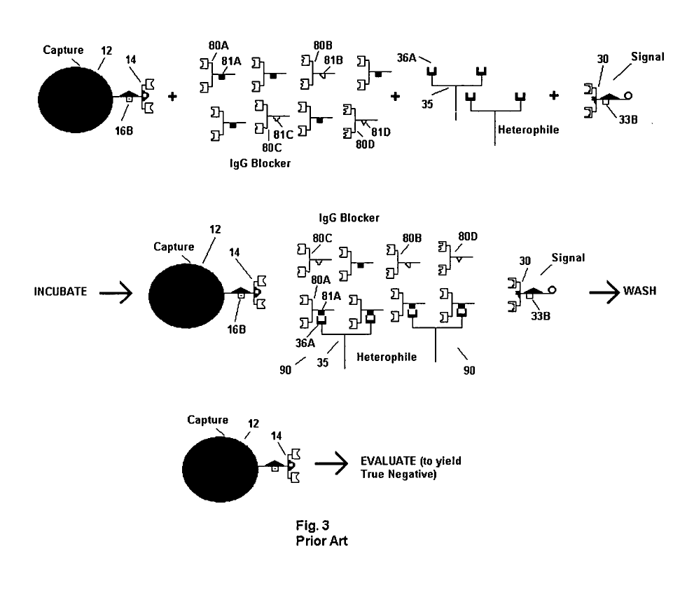

Obviously, either form of heterophile interference (false positive or negative) has an adverse effect on the accuracy, specificity or sensitivity of the immunoassay result. A typical prior art approach to eliminating the interfering effects of heterophile is provided in FIG. 3. This illustration provides a theoretical example of successful blocking of heterophiles, and as a result, minimization or reduction of the effects of heterophile interference.

In FIG. 3, the paramagnetic particle-supported specific antibody 14 is mixed with the target-specific signal antibody 30 and the patient sample comprising only heterophile molecules 35 and, in this case, no target analyte. In addition, an exemplary range of non-specific “normal IgG” antibodies 80A, 80B, 80C, 80D each with different binding regions 81A, 81B, 81C, 81D, respectively, are provided. Each of the binding regions 81A, 81B, 81C, 81D are illustrated as having distinct structural shapes indicating binding affinity for specific substances. The “normal IgG” antibodies 80A, 80B, 80C, 80D are added to the mixture to “flood” the patient sample in an effort to bind and block the problematic heterophiles. The heterophiles 35 in this exemplary case comprise binding regions 36A with a specific affinity and binding capability, illustrated by structural shape.

This mixture is incubated and complex 90 forms as desired, comprising a heterophile molecule 35 and one particular IgG antibody type 80A. Note that the particular heterophile 35 illustrated comprises a binding region 36A with specificity (indicated by complementary structural shaping) for heterophile binding region 16B on the capture antibody 14 and/or heterophile binding region 33B on the signal antibody 30. Thus, without the “normal IgG” blocker approach, the heterophiles 35 may interfere by binding either the capture antibody 14 or the signal antibody 30, or both. The IgG antibodies 80A act, at least theoretically, as a molecular sink in that they bind all heterophiles 35 with binding regions 36A, and thus prevent those particular heterophiles from otherwise interfering with the capture 14 or signal 30 antibodies.

Following washing, the heterophile complex 90 is eliminated along with other unbound material such as unbound IgGs 80B, 80C, 80D and the signal antibodies 30. In this exemplary case, since there was no target analyte present in the patient sample, the material remaining comprises the paramagnetic particle-supported capture antibody 14, without analyte or signal antibody 30. Thus, evaluation of the washed and retained material results in a true negative response.

Of course, in practice, the broad range IgG heterophile “blocking” approach provided in FIG. 3 is quite limited due to the non-specific nature of the IgG antibodies utilized. It is impossible using current techniques, for example, to ensure or even predict whether the correct form of IgG antibody is being used in combination with the specific antibodies employed in the immunoassay.

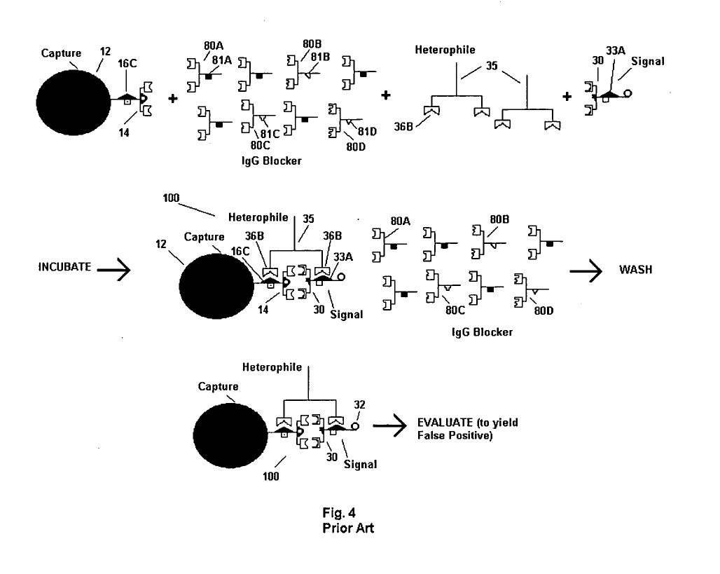

In contrast, FIG. 4 illustrates the limitations of the prior art IgG heterophile “flooding” blocking approach. Here, as in FIG. 3, the paramagnetic particle-supported specific antibody 14 is combined with the signal antibody 30 and the patient sample comprising heterophile molecules 35 having a particular binding region 36B and no analyte. In addition, a broad range of non-specific “normal IgG” antibodies 80A, 80B, 80C, 80D having binding regions 81A, 81B, 81C, and 81D, respectively, is added to the mixture and incubated.

Following incubation, complex 100 forms, comprising the specific capture antibody 14 and the signal antibody 30, bridged by a heterophile molecule 35, similar to the undesirable falsely positive complex formed in FIG. 2A. The binding regions 36B of the heterophile 35 bind with the heterophile binding regions 16C and 33A of the capture antibody 14 and the signal antibody 30, respectively.

Significantly, none of the IgG “blocker” binding regions 80A, 80B, 80C, and 80D on antibodies 80A, 80B, 80C and 80D, respectively, have affinity (again illustrated by complementary structural shapes) and binding capability for the heterophile binding regions 36B present in the exemplary patient sample. As a result, heterophiles 35 not bound by the IgG antibodies 80A, 80B, 80C, 80D are free to bind and interfere with the capture 14 and/or signal 30 antibodies and the “blocking” attempt fails.

Washing of the mixture results in retention of complex 100 and elimination of all other material. Evaluation of complex 100 yields a falsely positive result since the signal antibody 30, with signal-generating moiety 32 conjugated thereto, generates a signal in the complete absence of any analyte in complex 100.

A primary difficulty with the prior art “flooding” IgG blocker approach is that it cannot be predicted whether the result is inaccurately skewed in a falsely positive or negative direction since both forms of interference may occur simultaneously. Indeed, compounding the difficulty is that the nature of the molecules involved indicate that false positive and false negative results may occur simultaneously within the same assay. Thus, it is impossible to create an adjustment to compensate for the existence of such interference. Clearly, a more specific method and reagent for eliminating interfering substances, e.g., heterophile molecules, is needed.

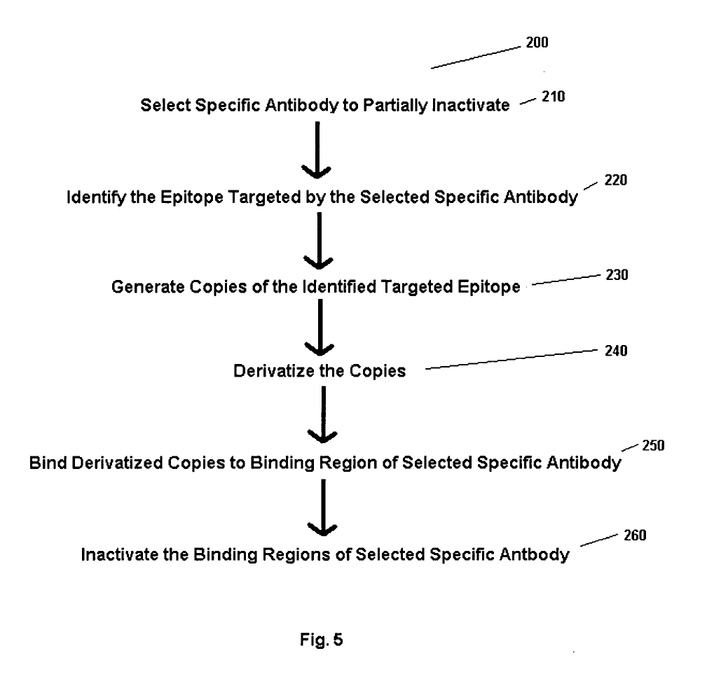

One embodiment of the inventive method of manufacturing a blocker reagent specifically designed to block, i.e., specifically bind and inactivate interfering substances such as heterophile molecules is provided in FIG. 5.

The method 200 begins with selection of a specific antibody 210. The antibody selected is an analyte-specific antibody used in the particular immunoassay. In the instance of a sandwich immunoassay, either the analyte-specific capture antibody or the analyte-specific signal antibody, or both, may be selected for use in the method.

Next, the epitopic structure, or binding region, on the target, e.g., analyte targeted by the selected specific antibody, is identified 220. This may be done by a number of methods known to those skilled in the art. For example, one approach may employ “epitope mapping”, in which oligopeptide fragments representing relevant amino acid sequences of the protein target are probed for their ability to interact with the specific antibody.

Once the targeted binding region structure is identified, the structure itself is copied 230. This is accomplished by any method well known to those skilled in the art. For example, one approach may employ solid phase peptide synthesis followed by HPLC purification, as needed.

The copies are next derivatized to yield reactive intermediate structures 240, by methods well known to those skilled in the art. In a preferred embodiment, a heterobifunctional reagent may be used wherein the copies are mixed with the heterobifunctional reagent to yield such reactive derivatized copies.

Next, the reactive derivatized copies from step 240 are bound to the analyte binding region of the selected specific antibody in step 250. In a preferred embodiment, the activated copies are mixed with the selected specific antibody from step 210. The resulting complex comprises the selected specific antibody with reactive copies bound to the analyte binding region(s) of the antibody.

Finally, the specific antibody selected in 210 is inactivated in step 260. In the preferred embodiment, the complex from step 250 is subjected to conditions that cause a reaction between the second moiety of the heterobifunctional reagent and the specific antibody, e.g., irradiated with light or other method as known to those skilled in the art. The inactivation reaction renders the target analyte binding regions inactivate, wherein the target analyte is prevented from binding thereto, but the heterophile interferent binding sites remain active.

Inactivation of the selected specific antibody or antibodies may occur by other methods, each of which is well within the scope of the invention. Generally, inactivation is achieved by at least one modification of the active binding region. For example, one or more specific amino acids in the analyte binding region may be modified by methods known to those skilled in the art, e.g., broad chemistry approaches. Alternatively, at least one substance may be bound with the selected antibody's analyte binding region(s). This substance bound with the analyte binding regions(s) may comprise at least one peptide. The peptide may be derivatized to inactivate the target analyte binding regions. Derivatization of the peptide may not be required, however, to achieve the desired inactivation. Another inactivation approach is to facilitate the binding of at least one derivatizing reagent, e.g., a heterobifunctional reagent or peptide, within the target analyte binding regions to achieve inactivation. The derivatizing reagent may further comprise at least one amino acid residue.

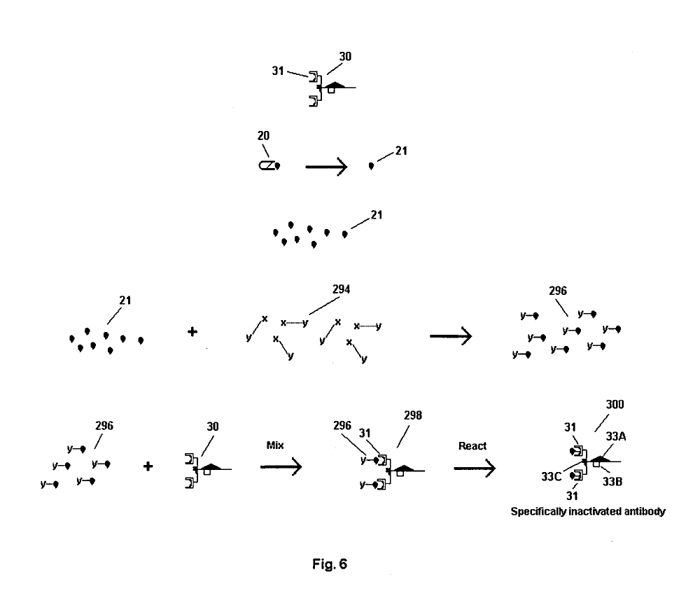

Turning now to FIG. 6, one embodiment of the method of manufacture is illustrated, resulting in the inventive blocker reagent.

Thus, the specific antibody is selected according to the preferred method. In the example, the specific signal antibody 30 in an immunoassay is the antibody chosen. Alternatively, the capture antibody may have been selected. In this particular instance, the antibody 30 has not been conjugated to include the signal element, though the signal element will not interfere with the blocking reagent function and may be present in other embodiments. Further, the selected antibody 30 comprises at least one analyte binding region 31, specific for the target or analyte's epitopic structure.

The epitopic structure 21 of the target analyte 20 is identified and copies are made of the identified binding region or epitopic structure 21. In a specific but not limiting example, the epitopic structure 21 can be an oligopeptide sequence within a polypeptide analyte 20.

The copies 21 are mixed with a derivatizing reagent 294 to provide reactive intermediates 296, capable of binding to the analyte binding region 31 of the selected antibody 30.

The reactive intermediates 296 are mixed with the active form of the selected specific antibody 30 to form a pre-blocker complex 298, wherein one reactive intermediate copy of the targeted epitopic structure 296 is bound to one of the specific antibody analyte binding regions 31. Preferably, all specific antibody analyte binding regions 31 bind a reactive target structure.

The pre-blocker complex 298 is then inactivated. Preferably, a second reaction, e.g., irradiation of a photoactivatable functionality with light, or other method as known to those skilled in the art, is used to complete the inactivation to produce the inventive specific blocker 300. The blocker's analyte-specific binding regions 31 are now inactive and cannot bind analyte 20; the heterophile interferent binding regions 33A, 33B, 33C remain active and capable of binding heterophile molecules.

It is understood that the inactivation of the target analyte binding region(s) of the selected specific antibody may result in the complete or partial inactivation of the analyte binding sites. Inactivation is defined in this context as inhibition of the binding of the analyte to the analyte binding region. Thus, “inactive” analyte binding regions cannot bind the targeted analyte.

Moreover, the inactivation method may change the structure of the selected specific antibody in some manner that does not affect the ability of the inactivated specific antibody to function as intended in binding with the heterophile molecules or other interfering substances. For example, the inactivation process may alter or otherwise affect the analyte binding site(s) and/or the heterophile binding site(s) or other regions of the inactivated antibody. In this connection, “active” heterophile binding site(s) are defined as retaining the ability to bind the heterophile, despite structural or other changes to the inactivated antibody. Thus, the structure of the inventive blocker may be identical, or substantially identical, to the structure of the active specific antibody and be within the scope of the present invention.

It is further understood that the analyte-specific binding region(s) of the blocker is inactivated while the interfering substance, e.g., heterophile, binding region(s) remain active. For descriptive purposes herein, the blocker is referred to herein as inactivated, even though the interfering substance, e.g., heterophile, binding region(s) remains active.

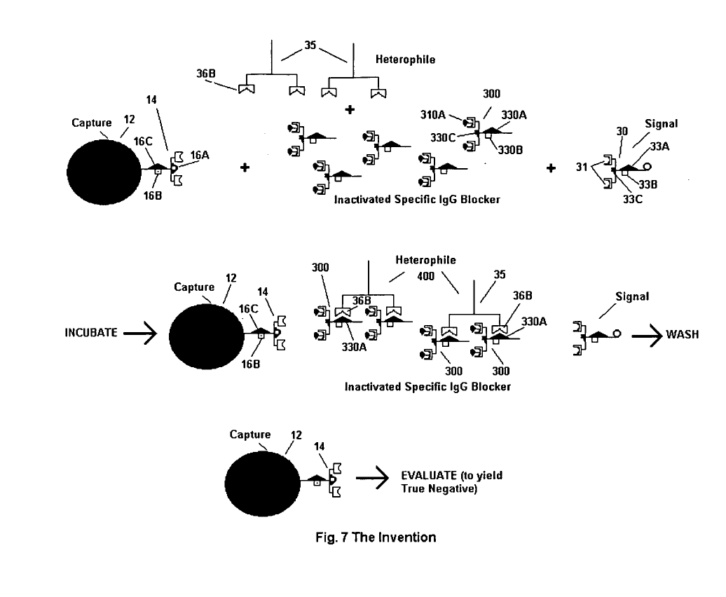

FIG. 7 provides one embodiment of the inventive blocker reagent used in an immunoassay reagent system and corresponding method to quantitatively or qualitatively analyze at least one target analyte in a test sample that may, or may not, contain interfering substances, e.g., heterophiles.

An amount of capture antibody 14, bound to the paramagnetic particle 12, is mixed with a patient sample comprising heterophiles 35 without analyte, the inventive blocker reagent 300, and the signal antibody 30. The capture antibody 14 comprises heterophile binding regions 16A, 16B, 16C, signal antibody 30 comprises heterophile binding regions 33A, 33B, 33C, and heterophile 35 comprises binding region 36B in this case. Binding region 36B is illustrated as having an affinity and ability to bind heterophile binding regions 16C and/or 33A. Significantly, heterophile binding region 36B may have capability to bind one or more binding regions on either the capture antibody 14, the signal antibody 30, or both.

Blocker reagent 300, an inactivated form of the selected specific antibody, in this case the signal antibody 30, is shown having three heterophile binding regions 330A, 330B and 330C, corresponding to heterophile binding regions 33A, 33B and 33C of the signal antibody 30. The signal antibody 30 comprises active analyte binding region(s) 31. The blocker 300 comprises inactive analyte binding regions(s) 310A. Thus, heterophiles 35 able to bind the blocker's heterophile binding region 330A (but not 330B or 330C) will bind thereto, while the analyte, when present, cannot bind to the analyte binding region(s) 310A. It is understood by those skilled in the art that the blocker reagent illustrated and described herein is one of many equivalent forms of molecules, all of which are well within the scope of the present invention.

The mixture is incubated and complex 400 forms as desired, comprising heterophile 35 bound to, in this exemplary embodiment, two of the inventive blocker molecules 300. Thus, heterophile binding region 36B is bound with the structurally corresponding heterophile binding region 330A on the blocker 300. Formation of complex 400 effectively and specifically blocks heterophiles 35 from affecting the immunoassay by binding in a specific manner the heterophiles that otherwise would interfere with either the capture antibody 14 and/or the signal antibody 30.

Following washing, all material not bound to a paramagnetic particle 12 is eliminated, including complex 400 and the signal antibody 30. Thus, the paramagnetic particle-supported capture antibody 14 remains for evaluation. Evaluation, in this case, yields a true negative result, either quantitative or qualitative, depending upon the type of evaluation utilized. Because there was no target analyte in the patient sample, the negative result is true and accurate.

FIG. 7 provides one embodiment wherein the blocker 300 is added simultaneously with the capture antibody 14, patient sample and signal antibody 30. Alternatively, the patient sample may be pretreated with the blocker 300 by premixing patient sample with blocker 300 prior to adding the capture antibody 14 and the signal antibody 30. This allows the blocker 300 to bind and inactivate any potentially interfering heterophiles before addition of the capture 14 and signal 30 antibodies, effectively eliminating competition between the heterophile/blocker, heterophile/capture antibody and heterophile/signal antibody binding possibilities.

The inventive blocker reagent and immunoassay reagent system and method is effective with all patient samples, including, inter alia, whole blood, plasma, serum, saliva, cerebral spinal fluid, urine, amniotic fluid, interstitial fluid, feces, mucus, cell extracts and tissue extracts. In addition, the blocker reagent and immunoassay method will work in any known immunoassay reagent system, including inter alia, nephelometric, turbidimetric and radiolabeled assay formats.

The blocker reagent described herein has broad application beyond reduction of interference in immunoassays. For example, monoclonal antibodies are used to enhance delivery of diagnostic and/or therapeutic agents to assess, diagnose, treat and/or prevent disease or a particular condition. By way of example, immunoconjugates, comprising an anticancer agent conjugated to a monoclonal antibody that is specific for a particular type of cancer cell, may be prepared and administered to the patient by known routes and methods. Significantly, immunoconjugates may comprise a number of diagnostic and/or therapeutic agents as readily recognized by those skilled in the art. Typical agents used against cancer cells are radioactive isotopes, drugs, toxins and enzymes.

The inventive blocker reagent may increase the efficacy of the exemplary anticancer immunoconjugate by administering a protective dose of blocking antibodies capable of highly specific binding with non-target cells, tissues and/or antigens encountered along the therapeutic pathway. The blocking reagent may be administered prior to the active immunoconjugate, simultaneously with, or in combination as a pharmaceutical composition. In this context, the non-target interfering substances, e.g., cells, tissues and/or antigens may comprise cells, combinations of cells, tissues, amino acids or functional constituent(s) or derivative(s) thereof, polypeptides, interfering antibodies, e.g., heterophile antibodies, and the like. The inventive blocker reagent may thus provide a highly specific protective dose that is designed to ensure to the extent possible, maximum dosage delivery of the active immunoconjugate agent at the target cell or tissue, by minimizing inactivating binding, interference, and or non-target retention of the immunoconjugate. The particular active agent or immunoconjugate protected by the blocker reagent is not, of course, limited to anticancer agents. Those skilled in the art will readily recognize the wide range of therapeutic and/or diagnostic agents that will benefit from use of the inventive blocker reagent.

The inventive blocker reagent when used as a protective dose as described herein may be administered prior to the active therapeutic and/or diagnostic agent or concurrently. If used as a component of a delivery vehicle for active agents, the blocker reagent may be associated with the active agent by any method known to those skilled in the art. By way of example, the active agent may be combined with the blocker reagent. In either case, a pharmaceutical composition comprising the blocker reagent and the active agent may be manufactured and administered to the subject.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. An immunoassay reagent system to qualitatively or quantitatively analyze at least one analyte in a test sample that might contain heterophile interferents, comprising:
at least one analyte-specific antibody; and
at least one heterophile blocker comprising an inactivated form of the at least one analyte-specific antibody, wherein the analyte is blocked from binding with the heterophile blocker.

2. The immunoassay reagent system of claim 1, wherein the at least one heterophile blocker further comprises at least one inactive analyte binding region, wherein the at least one analyte cannot bind with the blocker.

3. The immunoassay reagent system of claim 1, wherein the at least one heterophile blocker further comprises at least one active heterophile interferent binding region, wherein the at least one heterophile interferent may bind with the blocker.

4. The immunoassay reagent system of claim 1, wherein the at least one heterophile blocker further comprises at least one inactivated analyte binding region and at least one active heterophile interferent binding region.

5. The immunoassay reagent system of claim 2, wherein the at least one heterophile blocker further comprises at least one modification within the at least one analyte binding region to inactivate the at least one analyte binding region.

6. The immunoassay reagent system of claim 2, wherein the at least one heterophile blocker further comprises at least one substance bound with the at least one analyte binding region to inactivate the at least one binding region.

7. The immunoassay reagent system of claim 6, wherein the at least one substance further comprises at least one peptide to inactivate the at least one analyte binding region.

8. The immunoassay reagent system of claim 7, wherein the at least one peptide is derivatized.

9. The immunoassay reagent system of claim 6, wherein the at least one heterophile blocker further comprises at least one derivatizing reagent bound to the at least one binding region to inactivate the at least one binding region.

10. The immunoassay reagent system of claim 9, wherein the at least one derivatizing reagent comprises at least one amino acid residue.

11. A blocker reagent comprising an inactivated form of a target-specific antibody for use in reducing interference of the binding of a target analyte with the target-specific antibody or from the formation of a complex that would be formed in the absence of the target analyte causing a false positive result.

12. The blocker of claim 11, further comprising at least one inactive target analyte binding region, wherein the target analyte is blocked from binding with the blocker.

13. The blocker of claim 11, further comprising at least one active interfering substance binding region, wherein the at least one interfering substance can bind with the blocker.

14. The blocker of claim 12, further comprising at least one active interfering substance binding region, wherein the at least one interfering substance may bind with the blocker.

15. The blocker of claim 12, further comprising at least one modification within the at least one target analyte binding region to inactivate the at least one target analyte binding region.

16. The blocker of claim 12, further comprising at least one substance bound with the at least one target analyte binding region to inactivate the at least one target analyte binding region.

17. The blocker of claim 16, wherein the at least one substance further comprises at least one peptide to inactivate the at least one target analyte binding region.

18. The blocker of claim 12, wherein the target-specific antibody further comprises at least one derivatizing reagent bound with the at least one target analyte binding region to inactivate the at least one target analyte binding region.

19. The blocker of claim 18, wherein the at least one derivatizing reagent comprises at least one amino acid residue.

20. An immunoassay method for quantitatively or qualitatively analyzing at least one analyte in a test sample that may contain heterophilic interferents, comprising:
(a) forming a reaction complex by combining:
(i) a test sample;
(ii) at least one analyte-specific antibody; and
(iii) at least one heterophile blocker, wherein the blocker comprises an inactive form of the at least one analyte-specific antibody and wherein the at least one analyte-specific antibody is blocked from binding to the analyte in the test sample; and
(b) measuring the reaction complex to quantitatively or qualitatively determine the amount or presence of the analyte in the test sample.

21. The immunoassay method of claim 20, further comprising pretreating the test sample prior to forming the reaction complex to remove heterophile interferents by combining the at least one heterophile blocker with the test sample.

22. The immunoassay method of claim 20, wherein the test sample is selected from the group consisting of whole blood, plasma, serum, saliva, cerebral spinal fluid, urine, amniotic fluid, interstitial fluid, feces, mucus, cell extracts and tissue extracts.

23. The immunoassay method of claim 20, further comprising reducing the rate of false positive or false negative test results occurring from heterophilic interference.

24. The immunoassay method of claim 20, further comprising improving the sensitivity or specificity of the immunoassay by reducing heterophilic interference.

25. A method of manufacturing a heterophile interferent blocker reagent for use in an immunoassay reagent system, comprising:
a. selecting at least one antibody specific for an analyte;
b. determining the presence of at least one analyte binding region on the at least one antibody; and
c. inactivating the at least one analyte binding region, thereby blocking the at least one analyte from binding with the at least one analyte binding region.

26. The method of claim 25, wherein the inactivating further comprises modifying the at least one analyte binding region to block the at least one analyte from binding with the analyte binding region.

27. The method of claim 25, wherein the inactivating further comprises binding at least one substance to the at least one analyte binding region.

28. The method of claim 27, wherein the at least one substance comprises at least one peptide.

29. The method of claim 28, wherein the at least one peptide is derivatized.

30. The method of claim 27, wherein the at least one substance comprises at least one derivatizing reagent bound with the at least one analyte binding region.

31. The method of claim 30, wherein the at least one derivatizing reagent comprises at least one amino acid.

32. A method for improving delivery of at least one agent to a target in a subject, wherein at least one interfering substance interferes with delivery of the at least one agent to the target, comprising:
(a) providing at least one immunoconjugate, comprising the at least one agent and a target-specific antibody;
(b) preparing at least one specific blocker reagent that is an inactivated form of the target-specific antibody, wherein the target cannot bind, but the at least one interfering substance can bind;
(c) administering the at least one specific blocker reagent which, when bound with the at least one interfering substance, reduces or eliminates the interference; and
(d) administering the at least one agent concurrently with, or subsequent to, administering the blocker reagent.

33. The method of claim 32, further comprising:
combining the blocker reagent with the immunoconjugate to form a pharmaceutical composition; and
administering the pharmaceutical composition to the subject.

34. A pharmaceutical composition, the composition having a target, comprising:
at least one immunoconjugate comprising at least one target-specific antibody and at least one agent; and
at least one specific blocker, wherein the at least one specific blocker comprises an inactivated form of the at least one target-specific antibody.
